(19) United States
(12) Patent Application Publication
(10) Pub. No.: US 2007/0154466 A1
(43) Pub. Date: Jul. 5, 2007

(54) Internal medical devices containing peroxide-converting catalysts

(76) Inventors: Jan Weber, Maple Grove, MN (US); Eric D. Nielson, Bellevue, WA (US)

Correspondence Address: MAYER & WILLIAMS PC, 251 NORTH AVENUE WEST, 2ND FLOOR, WESTFIELD, NJ, 07090, US

(21) Appl. No.: 11/322,924

(22) Filed: Dec. 30, 2005

(51) Int. Cl. A61F 2/02; A61K 38/44; A61K 31/727

(52) U.S. Cl. 424/94.4; 424/426; 514/56

(57) ABSTRACT

The present invention is directed to implantable and insertable medical devices (also referred to herein as internal medical devices), which contain one or more peroxide-converting catalysts. According to an aspect of the invention, internal medical devices are provided, which contain (a) a substrate and (b) a peptide-containing, peroxide-converting catalyst disposed over at least a portion of the substrate. According to another aspect of the invention, internal medical devices are provided which contain a catalyst binding entity. The catalyst binding entity is adapted to bind to endogenous peroxide-converting catalyst upon implantation or insertion of the devices into patients. In another aspect of the invention, internal medical devices are provided which contain a biodegradable region that in turn contains a peroxide-converting catalyst. These devices release the catalyst in conjunction with degradation of the biodegradable region. In yet another aspect of the invention, internal medical devices are provided which contain a substrate, a multilayer region disposed over at least a portion of the substrate, and a peroxide-converting catalyst disposed within the multilayer region. The multilayer region, in turn, contains a plurality of charged layers of alternating charge, which charged layers further contain a plurality of polyelectrolyte containing layers.

### BACKGROUND OF THE INVENTION

High levels of hydrogen peroxide are harmful to tissue. In this regard, the use of catalytic metallic or ceramic stent coatings, such as iridium oxide coatings or platinum-enriched radiopaque stainless steel (PERSS) coatings, have shown some promise in reducing restenosis rates among patients.

Providing medical devices with metallic or ceramic surfaces having a high catalytic activity, however, may not be optimal with respect to other biochemical interactions and/or with respect to the mechanical properties of the devices. Furthermore, these catalytic surfaces need to be fluid-accessible in order to reduce the hydrogen peroxide levels. Hence, there is a design conflict if it is desired to apply non-permeable coatings over the catalytic surfaces.

### SUMMARY OF THE INVENTION

The above and other drawbacks are addressed by various aspects of the present invention, which is directed to implantable and insertable medical devices (also referred to herein as internal medical devices), which contain one or more peroxide-converting catalysts.

According to an aspect of the invention, internal medical devices are provided, which contain (a) a substrate and (b) a peptide-containing, peroxide-converting catalyst disposed over at least a portion of the substrate.

According to another aspect of the invention, internal medical devices are provided which contain a catalyst binding entity. The catalyst binding entity is adapted to bind to endogenous peroxide-converting catalyst upon implantation or insertion of the devices into patients.

In another aspect of the invention, internal medical devices are provided which contain a biodegradable region that in turn contains a peroxide-converting catalyst. These devices release the catalyst in conjunction with degradation of the biodegradable region.

In yet another aspect of the invention, internal medical devices are provided which contain a substrate, a multilayer region disposed over at least a portion of the substrate, and a peroxide-converting catalyst disposed within the multilayer region. The multilayer region, in turn, contains a plurality of charged layers of alternating charge, which charged layers further contain a plurality of polyelectrolyte containing layers.

The above and many other aspects, embodiments and advantages of the present invention will become immediately apparent to those of ordinary skill in the art upon review of the Detailed Description and Claims to follow.

### DETAILED DESCRIPTION OF THE INVENTION

According to an aspect of the present invention, implantable and insertable medical devices (also referred to herein as internal medical devices) are provided, which include one or more regions containing one or more peroxide-converting catalysts disposed over at least a portion of an underlying substrate. Here, the term “substrate” refers to a solid material region of the medical device.

Examples of internal medical devices for the practice of the present invention include, for example, stents (including coronary vascular stents, cerebral, urethral, ureteral, biliary, tracheal, gastrointestinal and esophageal stents), stent grafts, catheters (e.g., renal or vascular catheters such as balloon catheters), guide wires, balloons, filters (e.g., vena cava filters), cerebral aneurysm filler coils (including Guglilmi detachable coils and metal coils), vascular grafts, myocardial plugs, patches, pacemakers and pacemaker leads, heart valves, vascular valves, tissue engineering scaffolds for cartilage, bone, skin and other in vivo tissue regeneration, as well as any other device that is implanted or inserted into the body.

The medical devices of the present invention include medical devices that are used for diagnostics, systemic treatment, or for the localized treatment of any mammalian tissue or organ. Examples include tumors; organs including the heart, coronary and peripheral vascular system (referred to overall as “the vasculature”), lungs, trachea, esophagus, brain, liver, kidney, bladder, urethra and ureters, eye, intestines, stomach, pancreas, ovary, and prostate; skeletal muscle; smooth muscle; breast; dermal tissue; cartilage; and bone. As used herein, “treatment” refers to the prevention of a disease or condition, the reduction or elimination of symptoms associated with a disease or condition, or the substantial or complete elimination a disease or condition. Typical subjects (also referred to herein as “patients”) are mammalian subjects, more typically human subjects.

Substrates over which the catalyst-containing regions are disposed may be formed from metallic materials, as well as non-metallic materials including ceramic and polymeric materials. Thus, substrates for use in the present invention include those formed using one or more of the following: metal alloys such as cobalt-chromium alloys, nickel-titanium alloys (e.g., nitinol), cobalt-chromium-iron alloys (e.g., elgiloy alloys), nickel-chromium alloys (e.g., inconel alloys), and iron-chromium alloys (e.g., stainless steels, which contain at least 50% iron and at least 11.5% chromium), noble metals such as silver, gold, platinum, palladium, iridium, osmium, rhodium, and ruthenium, refractory metals such as titanium, tungsten, tantalum, zirconium, and niobium ,and bioabsorbable metals like magnesium and iron, as well as their alloys with one or more of the following: calcium, cerium, lithium, zinc and zirconium.

Substrates for use in the present invention further include those formed using one or more of the following: metal oxides, including aluminum oxides and transition metal oxides (e.g., oxides of titanium, zirconium, hafnium, tantalum, molybdenum, tungsten, rhenium, and iridium); silicon-based ceramics, such as those containing silicon nitrides, silicon carbides and silicon oxides (sometimes referred to as glass ceramics); calcium phosphate ceramics (e.g., hydroxyapatite); and carbon-based, ceramic-like materials such as carbon nitrides.

In addition, substrates for use in the present invention include those formed using one or more polymer species. As is well known, “polymers” are molecules that contain multiple copies of one or more types of constitutional units, commonly referred to as monomers, and typically containing from 5 to 10 to 25 to 50 to 100 to 500 to 1000 or more of each type of constitutional units. Polymers include, for example, homopolymers, which contain multiple copies of a single type of constitutional unit, and copolymers, which contain multiple copies of at least two dissimilar types constitutional units, which units may be present in any of a variety of distributions and include random copolymers, statistical copolymers, gradient copolymers, periodic copolymers (e.g., alternating copolymers), and block copolymers, among others. Polymers may have a variety of architectures, including cyclic, linear and branched architectures. Branched architectures include star-shaped architectures (e.g., architectures in which three or more chains emanate from a single molecular region), comb architectures (e.g., architectures having a main chain and a plurality of side chains) and dendritic architectures (e.g., arborescent and hyperbranched polymers), among others.

Hence, polymers for use in forming substrates and other components of the medical devices of the present invention (including coating layers as discussed below) may vary widely and include, for example, suitable members selected from the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides and polyether block amides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinyl acetate copolymers (EVA), polyvinylidene chlorides, polyvinyl ethers such as polyvinyl methyl ethers, polystyrenes, styrene-maleic anhydride copolymers, vinyl-aromatic-olefin copolymers, including styrene-butadiene copolymers, styrene-ethylene-butylene copolymers (e.g., a polystyrene-polyethylene/butylene-polystrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrenebutadiene copolymers and styrene-isobutylene copolymers (e.g., polyisobutylene-polystrene and polystyrene-polyisobutylene-polystyrene block copolymers such as those disclosed in U.S. Pat. No. 6,545,097 to Pinchuk), polyvinyl ketones, polyvinylcarbazoles, and polyvinyl esters such as polyvinyl acetates; polybenzimidazoles; ethylene-methacrylic acid copolymers and ethylene-acrylic acid copolymers, where some of the acid groups can be neutralized with either zinc or sodium ions (commonly known as ionomers); polyalkyl oxide polymers and copolymers including polyethylene oxides (PEO); polyesters including polyethylene terephthalates and aliphatic polyesters such as polymers and copolymers of lactide (including d-,l- and meso lactide), epsilon-caprolactone, glycolide, hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF), including elastomeric copolymers of vinylidene fluoride and hexafluoropropylene; silicone polymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof; examples of commercially available polyurethane copolymers include Bionate®, Carbothane®, Tecoflex®, Tecothane®, Tecophilic®, Tecoplast®, Pellethane®, Chronothane® and Chronoflex®); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends and further copolymers of the above.

In certain embodiments of the invention, the peroxide-converting catalysts may include one or more peptide-containing, peroxide-converting catalysts, which peptides may be, for example, full length enzymes or enzymatically active fragments thereof. In this regard, enzymes are found throughout the body and are among the fastest, most selective catalysts known to man. Peroxides, including hydrogen peroxide, are also found within the body, and there are a number of enzymes within the body that are able to reduce peroxide concentrations by converting them into non-peroxide reaction products. These include catalases, peroxidases (including glutathione peroxidases), peroxiredoxins, thioredoxin-linked systems, as well as derivatives, analogs and mixtures of the same.

In other embodiments, the peroxide-converting catalysts may include particles that contain one or more peroxide-converting metals and/or metal oxides such as a platinum group metal (e.g., platinum, iridium, osmium, palladium, rhodium, and ruthenium), a platinum group metal oxide, and mixtures thereof (e.g., a mixture of iridium oxide and platinum, among others).

The degree to which the peroxide-converting catalysts in the medical devices of the invention remain associated with the devices will depend, for example, upon the interactions between the peroxide-converting catalysts and the devices. These interactions may include covalent interactions, in which a chemical bond must be broken for release, and non-covalent physico-chemical interactions such as charge-charge interactions, charge-dipole interactions, dipole-dipole interactions including hydrogen bonding, charge-induced dipole interactions, Van der Waals interactions, hydrophobic interactions, physical entrapment/encapsulation, and combinations thereof.

Taking as examples peroxide-converting peptides, specifically enzymes (including whole enzymes and active enzyme fragments), such enzymes may be bound by adsorption to various material regions, both metallic and non-metallic. For example, it is known that enzymes readily adsorb to a number of materials, including aluminum oxide, controlled pore glass, and a variety of natural and synthetic polymers. Enzymes may be also be ionically bound to charged materials. Examples of such materials include natural polymers such as polysaccharides and synthetic polymers having ion-exchange centers.

Non-covalent binding may be specific to the enzyme of interest. Examples of binding mechanisms which provide good binding specificity include the following: binding based on the formation of multiple hydrogen bonds (e.g., analogous to base pairing), binding based on the formation of complexes and/or coordinative bonds, binding based on antibody-antigen interactions, also sometimes referred to as antibody-hapten interactions, (e.g., using whole antibodies or functional antibody fragments), protein-small molecule interactions (e.g., avidin/streptavidin-biotin binding), and so forth.

Many of these techniques require the attachment of a binding moiety to the substrate material region. In some instances, binding moieties are also attached to the enzyme to be bound (e.g., biotin may be attached to the enzyme), while in other instances binding moieties need not be attached (e.g., where an enzyme-specific antibody is attached to the substrate).

Where modification of the enzyme is not required for attachment (e.g., where a substrate is provided with an attached enzyme binding motif, such as an antibody or fragment thereof), the device may be capable of taking on peroxide-converting enzymes that are present in vivo (i.e., endogenous enzymes), after placement within a patient. These devices may be advantageous, for example, where the enzyme is not particularly robust under conditions encountered ex vivo, for example, during medical device sterilization.

Techniques are known by which catalase and other enzymes may be isolated, after which monoclonal antibodies may be generated. See, e.g., Wiemer EA, et al., “Production and characterisation of monoclonal antibodies against native and disassembled human catalase,” J. Immunol. Methods. 1992 Jul. 6;151(1-2):165-75; Jin LH, et al., “Human liver catalase: cloning, expression and characterization of monoclonal antibodies,” Mol Cells. 2003 Jun. 30;15 (3):381-6. Once generated, these antibodies may subsequently be attached to a substrate (also referred to as immobilization), for example, using various known covalent binding methods, including those discussed below.

Other methods for attaching peroxide-converting catalysts involve the formation of covalent bonds between the peroxide-converting catalyst and a substrate. Where covalent binding techniques are employed, the peroxide-converting catalyst is typically not released into the surrounding media, or it is only released upon cleavage of the covalent bond(s) holding the peroxide-converting catalyst to the substrate or upon breakdown of the substrate (e.g., in the case of a biodegradable polymeric substrate layer). Functional groups found on enzymes that may take part in covalent binding include, for example, amino, carboxyl, sulfhydryl, hydroxyl, imidazole, phenolic, thiol, threone and indole groups.

Covalent attachment to a substrate should involve only functional groups of the enzyme that are not essential for catalytic action. Thus, in general, higher activities result from prevention of reactions with amino acid residues of the active sites. The availability of a wide variety of covalent binding reactions and a wide variety of substrates (e.g., those with functional groups that are capable of participation in covalent coupling reactions or are capable of being activated to provide such groups) increases the likelihood that a suitable linking method will be found for a given enzyme where unacceptable losses in enzymatic activity do not occur. Also, a number of protective methods have been devised to prevent unacceptable losses in enzymatic activity, including covalent attachment of the enzyme in the presence of a competitive inhibitor or substrate, the formation of reversible covalently-linked enzyme-inhibitor complexes, the formation of chemically modified enzymes where covalent linkage to the substrate is achieved by newly incorporated residues, the use of zymogen precursors, and so forth.

Covalent coupling between peroxide-converting enzymes and substrates may proceed, for example, by direct reaction of functional groups found on the enzymes with those found on the substrates, or by using linking agents that contain reactive moieties capable of reaction with such functional groups. A few examples of known covalent reactions include diazotization reactions, amide bond formation, alkylation and arylation. reactions, Schiffs base formation, amidation reactions, thiol-disulfide interchange, bifunctional reagent binding, and so forth.

Specific examples of commonly used bifunctional coupling agents include glutaraldehyde, diisocyanates, diiosothiocyanates, bis(hydroxysuccinimide)esters, maleimidehydroxysuccinimide esters, carbodiimides, N,N′-carbonyldiimidazole imidoesters, and difluorobenzene derivatives, among others. One of ordinary skill in the art will recognize that any number of other coupling agents may be used depending on the functional groups present. In some embodiments, it is desirable for the substrate and the peroxide-converting catalyst to have differing functional groups, so as to avoid self-coupling reactions. Functional groups present on the peroxide-converting catalyst and/or substrate may be converted, as desired, into other functional groups prior to reaction, e.g., to confer additional reactivity or selectivity. Further information on covalent coupling may be found, for example, in U.S. Pub. No. 2005/0002865, which is incorporated by reference.

Published methods for covalent and non-covalent immobilization of catalase include the following: (a) catalase supported on porous alumina, as described in Vasudevan PT and Thakur DS, “Soluble and immobilized catalase. Effect of pressure and inhibition on kinetics and deactivation.,” Appl. Biochem. Biotechnol. 1994 Decemeber;49(3):173-89, (b) catalase immobilized on Sephadex-100, DEAE-Sephadex and polyvinyl alcohol supports by modification with 2-amino-4,6-dichloro-s-triazine followed by diaminohexane and glutaraldehyde as a crosslinking agent, prior to coating with gelatine, as described in Tarhan L., “Enzymatic properties of immobilized catalase on protein coated supports,” Biomed Biochim. Acta. 1990;49(5):307-16, (c) catalase immobilized via biotin-streptavidin (SA) cross-linker, for example, using techniques analogous to those used in Muzykantov VR, “Conjugation of catalase to a carrier antibody via a streptavidin-biotin cross-linker,” Biotechnol. Appl. Biochem. 1997 October;26 (Pt 2):103-9, (d) immobilization of catalase on antibodies adsorbed on carbon fabric is described in Litvinchuk AV, et al., Prikl. Biokhim. Mikrobiol. 1994 July-October; 30(4-5):572-81 (Article in Russian, abstract available on Medline), (e) catalase immobilized on poly(acrylic acid-co-vinyl alcohol) through amidation of the enzyme's terminal amine groups with the lateral carboxylic group of the polymer support, activated by dicycloxexyl carbodiimide, as described in Marcel Popa et al., “Catalase Immobilized on Poly(Acrylic Acid-co-Vinyl Alcohol),” Eurasian Chemico-Technological Journal 2002 4 (3): 199-206 and (f) immobilization of multi-subunit catalase using highly activated glyoxyl agarose followed by cross-linking with dextran-aldehyde, as described in Betancor L, et al., “Preparation of a stable biocatalyst of bovine liver catalase using immobilization and postimmobilization techniques,” Biotechnol. Prog. 2003 May-June; 19(3):763-767.

In certain embodiments of the invention, internal medical devices are provided which comprise (a) at least one polymeric region disposed over at least a portion of an underlying substrate and (b) at least one peroxide-converting catalyst associated with the polymeric region. By associating the peroxide-converting catalyst with the polymeric region (e.g., a polymeric coating), the underlying substrate (e.g., a metallic stent such as a stainless steel or nitinol stent, etc.) may be optimized in regards to biocompatibility or mechanical properties (e.g., flexibility, etc.), without the constraint of having to provide catalytic activity.

“Polymeric” regions are regions that contain polymers, and commonly contain 50 wt % to 75 wt % to 90 wt % to 95 wt % to 97.5 wt % to 99 wt %, or even more polymers. Polymers for forming such polymeric regions may be selected, for example, from those described above.

One or more peroxide-converting catalysts may be associated with a given polymeric region, for example, (a) by being provided within or beneath the polymeric region, in which case the polymeric region may, for example, regulate release of the peroxide-converting catalyst and/or transport of peroxide species to the peroxide-converting catalyst, and/or (b) by being bound to the surface of the polymeric region, for example, based on one or more covalent or non-covalent binding mechanisms such as those described above. For example, a peroxide-converting catalyst may be covalently bound to one or more polymer species within the polymeric region, or it may be non-covalently bound to the polymeric region, for instance, because it has an affinity for one or more polymers within the polymeric region or because is modified to have an affinity for such polymers. As a specific example of the latter, a hydrophilic peroxide-converting catalyst such an enzyme may be modified by providing it with a hydrophobic tail in order to improve the retention of the enzyme on and/or within a hydrophobic polymeric coating.

There is a growing literature showing that hydrogen peroxide may be used within the body as an inter-and intra-cellular signaling molecule. Hence, placing a permanent source of peroxide-converting catalyst in the body, while having a positive short term effect (e.g., by reducing elevated hydrogen peroxide levels, which may arise, for example, from stresses caused by initial placement of a medical device), may also have a negative long-term effect (e.g., by disruption of beneficial inter- and intra-cellular signaling).

Consequently, in certain embodiments, the medical devices of the invention undergo a decrease in catalytic activity over time, for instance, by releasably disposing at least a portion of the peroxide-converting catalyst within the devices. As previously indicated, the degree to which the peroxide-converting catalysts in the medical devices of the invention remain associated with the devices will depend, inter alia, upon the interactions between the peroxide-converting catalysts and the devices. As an example, the peroxide-converting catalyst may be non-covalently associated with the medical device such that the peroxide-converting catalyst is released over time (e.g., the peroxide-converting catalyst may be reversibly adsorbed to a device surface, it may be releasably disposed within a polymeric region of the device, for instance, by diffusion through the polymeric region, and so forth).

As another example, the peroxide-converting catalyst may be non-covalently or covalently associated with a biodegradable coating (e.g., by covalently binding it to a molecular component of the coating such as a biodegradable polymer molecule, or by trapping it within or beneath the biodegradable coating), with the result being that at least a portion of the peroxide-converting catalyst departs the device (with or without an attached breakdown product) upon biodegradation of the coating. For instance, a peroxide-converting enzyme, or particles of a peroxide-converting catalytic metal or metal oxide (e.g., platinum or iridium oxide particles), may be disposed within a biodegradable polymeric coating, beneath the polymeric coating, or both, such that the catalysts are released upon biodegradation of the polymeric coating.

Various biodegradable polymers are known in the art including polymers and copolymers of the following: lactide (including d-,l- and meso lactide), glycolide, epsilon-caprolactone, hydroxybutyrate, hydroxyvalerate, para-dioxanone, trimethylene carbonate (and its alkyl derivatives), 1,4-dioxepan-2-one, 1,5-dioxepan-2-one and 6,6-dimethyl-1,4-dioxan-2-one, as well as desaminotyrosine polyarylates, desaminotryrosine polycarbonates, polyanhydrides, PEG-polybutyl terephthalates, polyesteramides, and biodegradable polyurethanes such as poly(ester urethanes), among various others.

In some embodiments, the medical devices of the present invention include at least one multilayer region disposed over at least a portion of an underlying substrate, wherein at least one peroxide-converting catalyst is disposed beneath or within the multilayer region.

In certain of these embodiments, the multilayer regions contain a plurality of alternating, oppositely charged layers, for example, containing (a) a plurality of layers (e.g., 2 to 3 to 4 to 5 to 10 to 20 to 50 to 100 or more layers) which contain one or more polyelectrolyte species that are capable of providing the layers with an overall positive surface charge and (b) (a) a plurality of layers (e.g., 2 to 3 to 4 to 5 to 10 to 20 to 50 to 100 or more layers) which contain one or more polyelectrolyte species that are capable of providing the layers with an overall negative surface charge.

A wide variety of polyelectrolyte species are available for use in forming such charged layers. Polyelectrolytes are polymers having charged (e.g., ionically dissociable) groups. Usually, the number of these groups in the polyelectrolytes is so large that the polymers are soluble in polar solvents (including water) when in ionically dissociated form (also called polyions). Depending on the type of dissociable groups, polyelectrolytes may be classified as polyacids and polybases. When dissociated, polyacids form polyanions, with protons being split off. Polyacids include inorganic, organic and bio-polymers. Examples of polyacids are polyphosphoric acids, polyvinylsulfuric acids, polyvinylsulfonic acids, polyvinylphosphonic acids and polyacrylic acids. Examples of the corresponding salts, which are also called polysalts, are polyphosphates, polyvinylsulfates, polyvinylsulfonates, polyvinylphosphonates and polyacrylates. Polybases, on the other hand, contain groups which are capable of accepting protons, e.g., by reaction with acids, with a salt being formed. Examples of polybases having dissociable groups within their backbone and/or side groups are polyallylamine, polyethylimine, polyvinylamine and polyvinylpyridine. By accepting protons, polybases form polycations. Some polyelectrolytes have both anionic and cationic groups, but nonetheless have a net positive charge (in which case they are referred to herein as “polycations”) or negative charge (in which case they are referred to herein as “polyanions”), which may depend on the surrounding pH.

Suitable polyelectrolytes for use in the invention include those based on biological polymers and those based on synthetic polymers. Linear or branched polyelectrolytes can be used. Using branched polyelectrolytes can lead to less compact polyelectrolyte multilayers having a higher degree of wall porosity. Suitable polyelectrolytes include, for example, relatively low-molecular weight polyelectrolytes (e.g., polyelectrolytes having molecular weights of a few hundred Daltons) up to macromolecular polyelectrolytes (e.g., polyelectrolytes of biological origin, which commonly have molecular weights of several million Daltons).

Specific examples from which polyanions suitable for the practice of the present invention may be selected include the following: polyamines, including polyamidoamines, poly(amino methacrylates) including poly(dialkylaminoalkyl methacrylates) such as poly(dimethylaminoethyl methacrylate) and poly(diethylaminoethyl methacrylate), polyvinylamines, polyvinylpyridines including quaternary polyvinylpyridines such as poly(N-ethyl-4-vinylpyridine), poly(vinylbenzyltrimethylamines), polyallylamines such as poly(allylamine hydrochloride) (PAH), poly(diallyidialklylamines) such as poly(diallyidimethylammonium chloride), spermine, spermidine, hexadimethrene bromide (polybrene), polyimines including polyalkyleneimines such as polyethyleneimines, polypropyleneimines and ethoxylated polyethyleneimines, polycationic peptides and proteins, including histone polypeptides and polymers containing lysine, arginine, omithine and combinations thereof including poly-L-lysine, poly-D-lysine, poly-L,D-lysine, poly-L-arginine, poly-D-arginine, poly-D,L-arginine, poly-L-ornithine, poly-D-ornithine, poly-L,D-ornithine, gelatin, albumin, protamine (e.g., protamine sulfate), polycationic polysaccharides such as cationic starch and chitosan, polynucleotides such as DNA, as well as copolymers, derivatives and combinations of the preceding, among various others.

Specific examples from which polycations suitable for the practice of the present invention may be selected include the following: (a) polysulfonates including polyvinylsulfonates, poly(styrenesulfonates) such as poly(sodium styrenesulfonate) (PSS), sulfonated poly(tetrafluoroethylene), sulfonated polymers such as those described in U.S. Pat. No. 5,840,387, including sulfonated styrene-ethylene/butylene-styrene triblock copolymers, sulfonated styrenic homopolymers and copolymer such as a sulfonated versions of the polystyrene-polyolefin copolymers described in U.S. Pat. No. 6,545,097 to Pinchuk et al., which polymers may be sulfonated, for example, using the processes described in U.S. Pat. No. 5,840,387 and U.S. Pat. No. 5,468,574, as well as sulfonated versions of various other homopolymers and copolymers; (b) polysulfates such as polyvinylsulfates, sulfated and non-sulfated glycosaminoglycans as well as certain proteoglycans, for example, heparin, heparin sulfate, chondroitin sulfate, keratan sulfate, dermatan sulfate; (c) polycarboxylates such as acrylic acid polymers and salts thereof (e.g., ammonium, potassium, sodium, etc.), for instance, those available from Atofina and Polysciences Inc., methacrylic acid polymers and salts thereof (e.g., EUDRAGIT, a methacrylic acid and ethylacrylate copolymer), carboxymethylcellulose, carboxymethylamylose, and carboxylic acid derivatives of various other polymers, polyanionic peptides and proteins such as glutamic acid polymers and copolymers, aspartic acid polymers and copolymers, polymers and copolymers of uronic acids such as mannuronic acid, galatcuronic acid and guluronic acid, and their salts, for example, alginic acid and sodium alginate polyanions, hyaluronic acid polyanions, gelatin, and carrageenan polyanions; (d) polyphosphates such as phosphoric acid derivatives of various polymers; (e) polyphosphonates such as polyvinylphosphonates; (f) as well as copolymers, derivatives and combinations of the preceding, among various others.

Multilayer regions for use in the present invention can be assembled using layer-by-layer techniques. Layer-by-layer techniques can be used to coat a wide variety of substrates via electrostatic self-assembly. In the layer-by-layer technique, a first layer having a first surface charge is typically deposited on an underlying substrate, followed by a second layer having a second surface charge that is opposite in sign to the surface charge of the first layer, and so forth. The surface charge on the outer layer is reversed upon deposition of each sequential layer. In general, the substrate is either inherently charged or is made to have a charge, for example, using the techniques discussed further below.

It is interesting to note that the layer-by-layer method is a wet-wet process in which no drying is needed in between the build up of layers. In this regard, the various charged layers may be applied by a variety of wet techniques. These techniques include, for example, spraying techniques, dipping techniques, roll and brush coating techniques, techniques involving coating via mechanical suspension, ink jet techniques, spin coating techniques, web coating techniques and combinations of these processes. The choice of the technique will depend on the requirements at hand. For example, dipping and spraying techniques (without masking) can be employed, for instance, where it is desired to apply the species to an entire substrate. On the other hand, masking, as well as roll coating, brush coating and ink jet printing can be employed, for instance, where it is desired to apply the species only certain portions of the substrate (e.g., in the form of a pattern).

In certain embodiments, the multilayer regions of the present invention may include one or more layers that contain one or more peroxide-converting catalyst species.

This may be achieved, for example, by introducing peroxide-converting catalysts into the multilayer regions as charged entities. In some embodiments, the peroxide-converting catalyst that is selected may have an inherent charge. For instance, where the peroxide-converting catalyst is an enzyme, the pH may be adjusted to ensure that it is sufficiently far from the isoelectric pH of the enzyme, such that it behaves as a polyelectrolyte with sufficient charge for layer-by-layer assembly.

In other embodiments, it may be beneficial to provide the peroxide-converting catalyst with a charge. For example, peroxide-converting catalysts may be conjugated to charged polycations or polyanions such as those above, thereby effectively converting them into polyelectrolyte-containing species which may participate in the layer-by-layer process.

In some embodiments, the multilayer regions of the present invention may include one or more layers that contain one or more charged particle species. These particles can vary widely in size, but typically have at least one dimension (e.g., the thickness for a plate, the diameter for a sphere or a fiber, etc.) that ranges from 10 microns to 1 micron to 100 nm or less.

Charged particles for use in these embodiments of the present invention may be, for example, catalytic particles such as catalytic metal (e.g., platinum) particles, catalytic metal oxide (e.g., iridium) particles, and/or other particles such as peroxide-converting enzyme crystals (e.g., catalase crystals), which particles' surfaces may be modified as needed to posses a charge that is sufficient to allow them to participate in the layer-by-layer process (e.g., by covalently or non-covalently bonding a charged species to them, or by encapsulating them in a charged species).

In this regard, charged particles may be provided in the form of capsules that contain alternating layers of polyanions and polycations, in which case the surface charge will depend upon the last layer deposited. If desired a charged or uncharged peroxide-converting catalyst may be provided within the core of the capsule. For example, see Caruso, F., et al., “Enzyme Encapsulation in Layer-by-Layer Engineered Polymer Multilayer Capsules,” Langmuir 2000, 16, 1485-1488, in which the encapsulation of enzyme such as catalase is achieved by the sequential adsorption of oppositely charged polyelectrolytes onto enzyme crystal templates, thereby providing capsules with high enzyme loading. See also I. L. Radtchenko et al., “A novel method for encapsulation of poorly water-soluble drugs: precipitation in polyelectrolyte multilayer shells,” International Journal of Pharmaceutics, 242 (2002) 219-223.

As previously noted, in various embodiments of the invention, it is desirable to ensure that at least a portion of the peroxide-converting catalyst is disposed for release from the device. With multilayer regions, this may be implemented, for example, through the use of porous structures.

Alternatively, this may be implemented, for example, through the use of biodegradable polyelectrolyte layers. Specific examples of biodegradable polyelectrolytes include chitosan and heparin, among many others. For instance, multilayer films which contain ordered layers of charged enzymes (e.g., catalase) (ensuring that the pH is at a sufficiently “non-isoelectric” point as indicated above) may be assembled by means of alternating electrostatic adsorption with a biodegradable polyelectrolyte of opposite charge (e.g., a polyanion such as chitosan or a polycation such as heparin), as the case may be. If desired, some of the enzyme layers may be substituted with a biodegradable polyelectrolyte of the same charge as the enzyme. Hence, as a specific embodiment, the device may be covered with layers of enzyme (e.g., catalase), polycation (e.g., heparin), and polyanion (e.g., chitosan). These layers will degrade/dissolve over time releasing both the catalase as well as the heparin.

The amount of enzyme incorporated, as well as the release profile for the enzyme, may be modulated, for example, by changing the size of the enzyme core and/or by changing the number of charged enzyme containing layers (depending, for instance, on whether the enzyme is provided as an encapsulated core material, is provided as a charged polyelectrolyte layer, is provided as a charged particle layer, or a combination of the same), by changing the number polyanion and/or polycation layers, by utilizing different polyanions and/or different polycations, and so forth.

A few specific examples include (a) a layering pattern of AC1AC2AC1A, where A is a polyanion layer, C1is a first polycation layer, C2 is a second differing polycation layer, (b) CA1CA2CA1C, where C is a polycation layer, A1 is a first polyanion layer, A2 is a second differing polyanion layer, (c) ACAP1ACP2CAC, where A is a polyanion layer, C is a polycation layer, P1is a first peroxide-converting catalyst layer (in this case, having a positive charge), P2 is a second peroxide-converting catalyst layer (in this case, having a negative charge). Clearly, the variants are essentially endless.

As indicated above, certain substrates are inherently charged and thus readily lend themselves to layer-by-layer assembly. To the extent that a substrate does not have an inherent net surface charge, a surface charge may nonetheless be provided. For example, where the substrate to be coated is conductive, a surface charge may be provided by applying an electrical potential to the same. Once a first polyelectrolyte layer is established in this fashion, a second polyelectrolyte layer having a second surface charge that is opposite in sign to the surface charge of the first polyelectrolyte layer can readily be applied, and so forth.

As another example, the substrate may be provided with a positive charge by covalently attaching entities with functional groups that have a positive charge (e.g., amine, imine or another basic groups) or a negative charge (e.g., carboxylic, phosphonic, phosphoric, sulfuric, sulfonic, or other acid groups) using coupling methods well known in the art, a few examples of which are described above in conjunction with enzyme immobilization.

In other examples, a surface charge is provided on a substrate by adsorbing polycations (e.g., protamine sulfate, polyallylamine, polydiallyldimethylammonium species, polyethyleneimine, chitosan, gelatin, spermidine, albumin, among many others) or by adsorbing polyanions (e.g., polyacrylic acid, sodium alginate, polystyrene sulfonate, eudragit, gelatin [gelatin is an amphiphilic polymer, hence it fits in both categories depending how it is being prepared], hyaluronic acid, carrageenan, chondroitin sulfate, carboxymethylcellulose, among many others) to the surface of the substrate as a first charged layer. Although full coverage may not be obtained for the first layer, once several layers have been deposited, a full coverage should ultimately be obtained, and the influence of the substrate is expected to be negligible. The feasibility of this process has been demonstrated on glass substrates using charged polymeric (polyelectrolyte) materials. See, e.g., “Multilayer on solid planar substrates,” Multi-layer thin films, sequential assembly of nanocomposite materials, Wiley-VCH ISBN 3-527-30440-1, Chapter 14; and “Surface-chemistry technology for microfluidics,” Hau, Winky L. W. et al. J. Micromech. Microeng. 13 (2003) 272-278.

For additional information on layer-by-layer assembly, see, e.g., U.S. Patent Appln. No. 2005/0037050, and references cited therein.

In some embodiments, the medical devices of the invention are optionally provided with one or more therapeutic agents (in addition to one or more peroxide-converting catalysts). “Therapeutic agents,” “drugs,” “bioactive agents” “pharmaceuticals,” “pharmaceutically active agents”, and other related terms may be used interchangeably herein and include genetic and non-genetic therapeutic agents. Therapeutic agents may be used singly or in combination.

For instance, the therapeutic agents may be associated with the medical devices of the invention using the techniques discussed above for associating catalyst molecules and particles with the devices. As an example, in certain embodiments, it may be desirable to include therapeutic agents within multilayer regions like those described above. As with the peroxide-converting catalysts, this may be done, for instance, by introducing the therapeutic agents into the multilayer regions as charged entities. Hence, a therapeutic agent may be selected that has an inherent charge (heparin is given above as an example), adjusting the pH as needed to provide sufficient charge. Alternatively, a therapeutic agent may be conjugated to charged polyions. Taking paclitaxel as a specific example, various ionic forms of paclitaxel are known, including paclitaxel-poly(l-glutamic acid) and paclitaxel-poly(1-glutamic acid)-PEO. In addition to these, U.S. Pat. No. 6,730,699, which is incorporated by reference in its entirety, also describes paclitaxel conjugated to various other polymers including poly(d-glutamic acid), poly(d-glutamic acid), poly(l-aspartic acid), poly(d-aspartic acid), poly(d-aspartic acid), poly(l-lysine), poly(d-lysine), poly(dl-lysine), copolymers of the above listed polyamino acids with polyethylene glycol, polycaprolactone, polyglycolic acid and polylactic acid, as well as poly(2-hydroxyethyl 1-glutamine), chitosan, carboxymethyl dextran, hyaluronic acid, human serum albumin and alginic acid. As another alternative, charged therapeutic particles may be provided, for example, by binding charged species to therapeutic agent particles (again using paclitaxel as a specific example, protein-bound paclitaxel particles such as albumin-bound paclitaxel nanoparticles, e.g., ABRAXANE are known) or by encapsulating them within charged species (e.g., using multilayer encapsulation techniques such as those described above).

A range of therapeutic agent loadings can be used in conjunction with the devices of the present invention, with the pharmaceutically effective amount being readily determined by those of ordinary skill in the art and ultimately depending, for example, the nature of the therapeutic agent itself, the environment into which the medical article is introduced, that nature of the association between the therapeutic agent and the device, and so forth.

Numerous therapeutic agents useful for the practice of the present invention may be selected from those described in paragraphs [0040] to [0046] of commonly assigned U.S. Patent Application Pub. No. 2003/0236514, the disclosure of which is hereby incorporated by reference. Examples include anti-thrombotic agents, anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms, among others.

A few specific beneficial therapeutic agents include vascular endothelial growth factors (e.g., VEGF-2), antithrombotic agents (e.g., heparin), sirolimus, paclitaxel (including particulate forms thereof such as ABRAXANE albumin-bound paclitaxel nanoparticles), everolimus, tacrolimus, Epo D, dexamethasone, estradiol, halofuginone, cilostazole, geldanamycin, ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, and Serca 2 gene/protein, resiquimod, imiquimod (as well as other imidazoquinoline immune response modifiers), human apolioproteins (e.g., AI, AII, AIII, AIV, AV, etc.), as well a derivatives of the forgoing, among many others.

Although various embodiments are specifically illustrated and described herein, it will be appreciated that modifications and variations of the present invention are covered by the above teachings and are within the purview of the appended claims without departing from the spirit and intended scope of the invention.

1. An implantable or insertable medical device comprising a substrate and peptide-containing, peroxide-converting catalyst disposed over at least a portion of said substrate.

2. The medical device of claim 1, wherein said device is a stent.

3. The medical device of claim 1, wherein said catalyst is selected from catalases, peroxidases, peroxiredoxins, thioredoxin-linked catalysts, catalytically active portions thereof, and mixtures thereof.

4. The medical device of claim 1, wherein said substrate is a metallic substrate.

5. The medical device of claim 1, wherein said catalyst is covalently bound to said substrate.

6. The medical device of claim 1, wherein said catalyst is non-covalently bound to said substrate.

7. The medical device of claim 1, wherein said device further comprises a polymeric region over said substrate.

8. The medical device of claim 7, wherein said catalyst is covalently or non-covalently bound to said polymeric region.

9. The medical device of claim 7, wherein said catalyst is provided within said polymeric region, beneath said polymeric region, or both.

10. An implantable or insertable medical device comprising a catalyst binding entity, said catalyst binding entity being adapted to bind to an endogenous peroxide-converting catalyst upon implantation or insertion of said device into a patient.

11. The medical device of claim 10, wherein said device is a stent.

12. The medical device of claim 10, wherein said endogenous peroxide-converting catalyst is selected from catalases, peroxidases, peroxiredoxins, thioredoxin-linked catalysts, catalytically active portions thereof, and mixtures thereof.

13. The medical device of claim 10, wherein said endogenous peroxide-converting catalyst comprises a catalase or a catalytically active portion thereof.

14. The medical device of claim 10, wherein said enzyme binding motif comprises an antibody or a portion thereof.

15. The medical device of claim 10, wherein said enzyme binding motif comprises an anti-catalyase antibody or a portion thereof.

16. An implantable or insertable medical device comprising a biodegradable region that comprises a peroxide-converting catalyst, wherein said device releases said catalyst in conjunction with biodegradation of said biodegradable region.

17. The medical device of claim 16, wherein said device is a stent.

18. The medical device of claim 16, wherein said biodegradable region comprises a catalyst selected from catalases, peroxidases, peroxiredoxins, thioredoxin-linked catalysts, catalytically active portions thereof, and mixtures thereof.

19. The medical device of claim 16, wherein said biodegradable region comprises catalytic particles.

20. The medical device of claim 19, wherein said catalytic particles comprise a catalyst selected from a peroxide-converting metal, a peroxide-converting metal oxide, and a mixture thereof.

21. The medical device of claim 19, wherein said catalytic particles comprise a catalyst selected from a platinum group metal, a platinum group metal oxide, and mixtures thereof.

22. The medical device of claim 16, wherein said biodegradable region is a biodegradable polymeric region.

23. The medical device of claim 22, wherein said catalyst is covalently bound to said polymeric region, non-covalently bound to said polymeric region, or both.

24. The medical device of claim 22, wherein said catalyst is provided within said polymeric region, beneath said polymeric region, or both.

25. An implantable or insertable medical device comprising a substrate, a multilayer region disposed over at least a portion of said substrate, and a peroxide-converting catalyst disposed within said multilayer region, said multilayer region comprising a plurality of charged layers of alternating charge, which further comprise a plurality of polyelectrolyte containing layers.

26. The medical device of claim 25, wherein at least one of said charged layers comprises charged particles that comprise a peroxide-converting catalyst.

27. The medical device of claim 26, wherein said particles comprise a peroxide-converting enzyme or a catalytically active portion thereof.

28. The medical device of claim 26, wherein said particles comprise a catalyst selected from catalases, peroxidases, peroxiredoxins, thioredoxin-linked catalysts, catalytically active portions thereof, and mixtures thereof.

29. The medical device of claim 26, wherein said particles comprise a peroxide-converting metal or a peroxide-converting metal oxide.

30. The medical device of claim 26, wherein said particles comprise iridium oxide or platinum metal.

31. The medical device of claim 25, wherein at least one of said polyelectrolyte containing layers comprises a polyelectrolyte species selected from polyethyleneimine, poly(allylamine hydrochloride), poly(diallyldialklylamine), chitosan, and combinations thereof.

32. The medical device of claim 25, wherein at least one of said polyelectrolyte containing layers comprises a polyelectrolyte species selected from poly(sodium styrenesulfonate), DNA, heparin, and combinations thereof.

33. The medical device of claim 25, wherein at least one of said charged layers comprises a peroxide-converting enzyme or a catalytically active portion thereof.

34. The medical device of claim 25, wherein a plurality of said charged layers comprise a peroxide-converting enzyme or a catalytically active portion thereof.

35. The medical device of claim 34, wherein said peroxide-converting enzyme is selected from catalases, peroxidases, peroxiredoxins, thioredoxin-linked catalysts, catalytically active portions thereof, and mixtures thereof.

36. The medical device of claim 34, wherein a plurality of said charged layers comprise a charged therapeutic agent.

37. The medical device of claim 34, wherein a plurality of said charged layers comprise heparin.

38. The medical device of claim 34, wherein said multilayer region comprises a plurality of charged layers that comprise heparin, a plurality of charged layers that comprise chitosan, and a plurality of charged layers that comprise catalase or a catalytically active portion thereof.
